(19) United States
(12) Patent Application Publication
(10) Pub. No.: US 2007/0255269 A1
(43) Pub. Date: Nov. 1, 2007

(54) Multi-channel radio frequency generator for high-frequency thermal treatment

(75) Inventor: Kyong-Min Shin, Seoul (KR)

Correspondence Address: LADAS & PARRY, 26 WEST 61ST STREET, NEW YORK, NY, 10023, US

(73) Assignees: TAEWOONG MEDICAL CO., LTD.; KYONG-MIN SHIN

(21) Appl. No.: 11/474,113

(22) Filed: Jun. 22, 2006

(30) Foreign Application Priority Data
Apr. 28, 2006 (KR) 10-2006-0038870

(51) Int. Cl. A61B 18/18; A61B 18/14

(52) U.S. Cl. 606/34; 606/38; 606/41; 606/45; 606/49

(57) ABSTRACT

A multi-channel radio frequency generator for high-frequency thermal treatment is used in combination with a plurality of electrodes. The multi-channel radio frequency generator includes an oscillator for producing a high-frequency, a waveform modulator part for modulating an waveform of the high-frequency generated by the oscillator, first to third amplifying channels for amplifying the high-frequency outputted from the waveform modulator part to have a root mean square output of 30-200 Watts and then supplying the root mean square output to the electrodes, a channel selection part for allowing a user to select one or more of the first to third amplifying channels, a key part for enabling the user to set a voltage, a current and an impedance for the first to third amplifying channels, and a microcontroller unit for controlling a power, a time and a phase of the high-frequency outputted from each of the first to third amplifying channels.

### FIELD OF THE INVENTION

The present invention is directed to a multi-channel radio frequency generator for high-frequency thermal treatment and, more specifically, to a multi-channel radio frequency generator capable of supplying a high-frequency to a plurality of electrodes to thereby efficiently fulgurate large-sized cancer cells occupying an increased area with enhanced safety and also capable of controlling more than one amplifying channels to perform simultaneous treatments for remotely located lesions.

### BACKGROUND OF THE INVENTION

Cancers caused in bodily organs such as a liver and the like are typically treated by a non-surgical method or a surgical operation. The surgical operation requires broad incision of the bodily portion at which a cancer lesion located, thus leaving an enlarged scar after the operation and necessitating a prolonged period of medical care. If the cancers recur in the bodily organ, the surgical operation has to be performed again. This is not only painful to a patient but also burdens the patient with heavy expenditure and risk.

For these reasons, non-surgical treatment methods are extensively used including, e.g., a transarterial chemoembolization, a percutaneous ethanol injection therapy, a systemic cancer chemotherapy and a local red heat treatment, among which the local red heat treatment has proven very effective. Examples of the local red heat treatment include a high-frequency heat treatment, a microwave cautery and a laser cautery, of which the high-frequency heat treatment is highly favored in recent years.

The high-frequency heat treatment refers to a method whereby the cancer cells created in, e.g., a liver, are fulgurated by cauterizing them with high-frequency heat without resort to incision. For the purpose of high-frequency heat treatment, there have been conventionally used a device of the type including a radio frequency generator for generating a high-frequency of constant voltage level and an electrode connected to the radio frequency generator.

Due to the fact that the conventional treatment device is comprised of a single radio frequency generator and a single electrode, the area of cauterization executable by the device is too narrow to fulgurate a large-sized cancer cell lump, thus making the treatment process time-consuming. Furthermore, the conventional treatment device cannot be used in treating remotely located lesions.

### SUMMARY OF THE INVENTION

Taking into account the above and other problems inherent in the prior art, it is an object of the present invention to provide a multi-channel radio frequency generator capable of supplying a high-frequency to a plurality of electrodes to thereby efficiently fulgurate large-sized cancer cells occupying an increased area with enhanced safety and also capable of controlling more than one amplifying channels to perform simultaneous treatments for remotely located lesions.

With this object in view, the present invention provides a multi-channel radio frequency generator for high-frequency thermal treatment used in combination with a plurality of electrodes, comprising: an oscillator for producing a high-frequency; a waveform modulator part for modulating a waveform of the high-frequency generated by the oscillator; first to third amplifying channels for amplifying the high-frequency outputted from the waveform modulator part to have a root mean square output of 30-200 Watts and then supplying the root mean square output to the electrodes; a channel selection part for allowing a user to select one or more of the first to third amplifying channels; a key part for enabling the user to set a voltage, a current and an impedance for the first to third amplifying channels; and a microcontroller unit for controlling a power, a time and a phase of the high-frequency outputted from each of the first to third amplifying channels.

### BRIEF DESCRIPTION OF THE DRAWINGS

The above and other objects, features and advantages of the present invention will become apparent from the following description of preferred embodiments given in conjunction with the accompanying drawings, in which:

FIG. 1 is a block diagram illustrating a multi-channel radio frequency generator for high-frequency thermal treatment in accordance with the present invention;

FIG. 2 shows a front panel of the multi-channel radio frequency generator in accordance with the present invention;

FIG. 3 illustrates one exemplary use of the multi-channel radio frequency generator shown in FIG. 1;

FIG. 4 shows a plurality of electrodes connected to the front panel of the multi-channel radio frequency generator in accordance with the present invention; and

FIG. 5 illustrates an instance where two multi-channel radio frequency generators are associated with each other and used in combination.

### DETAILED DESCRIPTION OF THE INVENTION

One preferred embodiment of a multi-channel radio frequency generator for high-frequency thermal treatment in accordance with the present invention will now be described in detail with reference to the accompanying drawings.

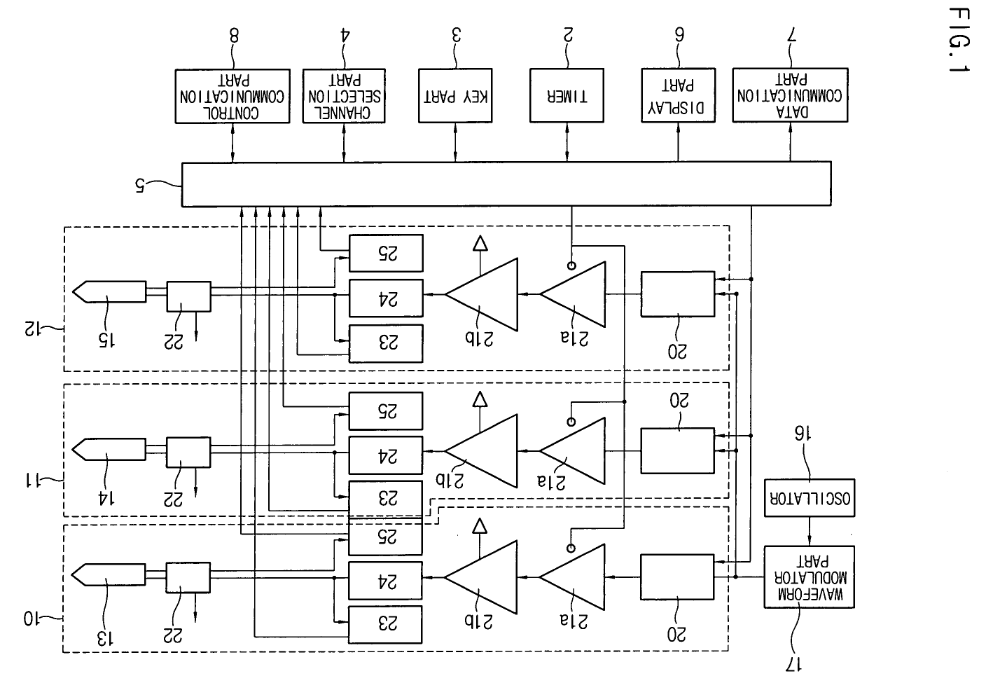

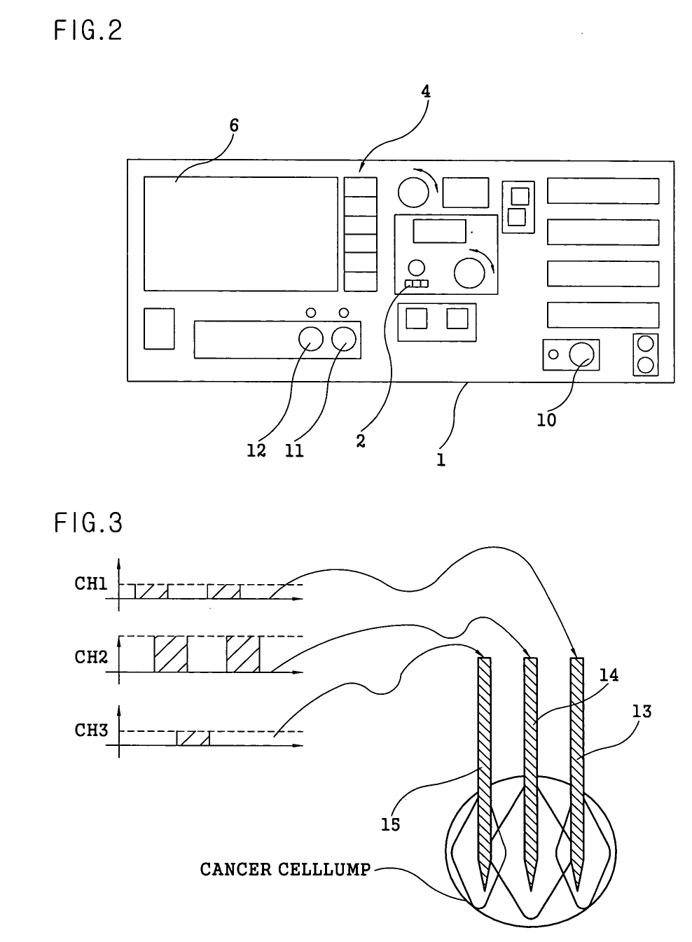

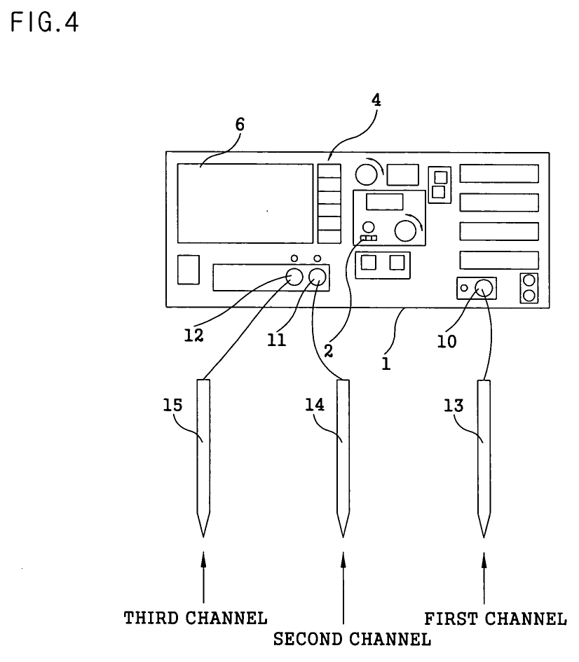

Referring FIGS. 1 through 5, a multi-channel radio frequency generator 1 is used in combination with a plurality of electrodes for high-frequency thermal treatment of, e.g., a cancer. The multi-channel radio frequency generator 1 includes an oscillator 16 for producing a high-frequency and a waveform modulator part 17 for modulating a waveform of the high-frequency generated by the oscillator 16.

First to third amplifying channels 10, 11 and 12 are connected to the waveform modulator part 17 for amplifying the high-frequency outputted from the waveform modulator part 17 to have a root mean square (RMS) output of 30-200 Watts and then supplying the root mean square output to the corresponding electrodes. It should be appreciated that the number of the amplifying channels is not restricted to three but may be greater or lesser in the present invention.

A channel selection part 4 is provided for allowing a user to select one or more of the first to third amplifying channels 10, 11 and 12, and a key part 3 is provided for enabling the user to set a voltage, a current and an impedance for the first to third amplifying channels 10, 11 and 12.

Also provided is a microcontroller unit 5 for controlling a power, a time and a phase of the high-frequency outputted from each of the first to third amplifying channels 10, 11 and 12.

Each of the first to third amplifying channels 10, 11 and 12 includes a phase control part 20 for controlling a phase of the high-frequency outputted from the waveform modulator part 17, a preamplifier 21a and a main amplifier 21b for amplifying in two stages the high-frequency outputted from the waveform modulator part 17 to have a root mean square output of 30-200 Watts and a load of about 50Ω, and a relay 22 for supplying the high-frequency outputted from the main amplifier 21b to the corresponding one of the electrodes 13, 14 and 15.

In addition, each of the first to third amplifying channels 10, 11 and 12 further includes a power sensor 23 for sensing a current and a voltage of the high-frequency supplied to the relay 22 and supplying the sensed current and voltage to the microcontroller unit 5, an impedance matching part 24 for matching an impedance on an output side of the main amplifier 21b with an impedance on a load side of the relay 22, and a temperature sensor 25 for sensing a temperature of the electrodes 13, 14 and 15 and supplying the sensed temperature to the microcontroller unit 5.

The microcontroller unit 5 is adapted to control amplification ratios of the preamplifier 21a and the main amplifier 21b in such a manner that the current and the voltage sensed by the power sensor 23 are kept from excessively increasing above predetermined values. The microcontroller unit 5 is also adapted to, in response to the temperature sensed by the temperature sensor 25, control the high-frequency outputted from each of the first to third amplifying channels 10, 11 and 12 in such a manner that the electrodes 13, 14 and 15 are operated within a temperature range of 10-90° C.±4.

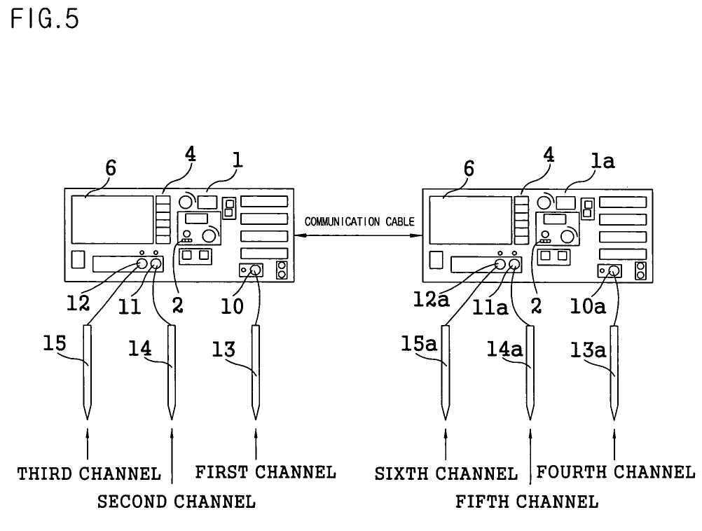

In addition to the above, the multi-channel radio frequency generator of the present invention further includes a timer 2 for setting and measuring an operation time of each of the first to third amplifying channels 10, 11 and 12, a display part 6 for displaying information detected by the power sensor 23 and the temperature sensor 25 of the first to third amplifying channels 10, 11 and 12 on a channel-by-channel basis, a data communication part 7 for notifying a remote computer of an operating condition of the radio frequency generator 1 so that the user can monitor the operating condition of the radio frequency generator 1, and a control communication part 8 to which one or more additional radio frequency generator 1a is connected for use in combination, as illustrated in FIG. 5.

In the case that one or more additional radio frequency generator 1a is connected to the control communication part 8, the multi-channel radio frequency generator 1 serves as a master generator, while the additional radio frequency generator is used as a slave generator, and vice versa.

Next, description will be given to the operation and advantageous effects of the multi-channel radio frequency generator set forth above. Operation of the inventive multi-channel radio frequency generator is divided into a single channel activation method in which only one of the first to third amplifying channels 10, 11 and 12 is activated and a multi-channel activation method in which more than one of them are activated for high-frequency thermal treatment.

[Single Channel Activation Method]

This method is used to fulgurate, e.g., cancer cells of a relatively small size, with a single electrode.

First of all, the user manipulates the key part 3 to set the voltage, current and impedance of the high-frequency which is to be supplied to the electrode 13 and then selects the first amplifying channel 10 with the use of the channel selection part 4.

Under this state, if a specific key of the key part 3 is pushed by the user, the oscillator 16 generates a high-frequency of, e.g., 480 KHz, which in turn is supplied to the waveform modulator part 17. The waveform modulator part 17 modulates the waveform of the high-frequency into a form suitable for use in the electrode 13 and then supplies the high-frequency to the phase control part 20 of the first amplifying channel 10. The phase control part 20 serves to adjust the phase of the high-frequency prior to the latter being supplied to subsequent elements.

The preamplifier 21a and the main amplifier 21b, which are serially connected to the phase control part 20, function to amplify the signals of the high-frequency to have a root mean square (RMS) output of, e.g., 30-200 Watts and a load of, e.g., about 50Ω. The signals of the high-frequency are then fed to the electrode 13 through the relay 22.

At this time, the microcontroller unit 5 controls the phase control operation of the phase control part 20 and the amplification ratio of the preamplifier 21a and the main amplifier 21b. The microcontroller unit 5 also controls the first amplifying channel 10 in such a manner that it can generate the output for a time period set and measured by the timer 2. The impedance matching part 24 disposed between the main amplifier 21b and the relay 22 serves to match the impedance on an output side of the main amplifier 21b with the impedance on a load side of the relay 22.

In this manner, the high-frequency is applied to the electrode 13 selected by the user. If the electrode 13 is kept in contact with or inserted into the lump of cancer cells, the cancer cells are fulgurated by the heat generated in the electrode 13.

Meanwhile, during the time when the high-frequency is supplied to the electrode 13, the power sensor 23 detects the current and voltage of the high-frequency and then feeds back the sensed current and voltage to the microcontroller unit 5. Furthermore, the temperature sensor 25 detects the temperature of the sensor 13 and then feeds back the sensed temperature to the microcontroller unit 5.

In addition, the microcontroller unit 5 keeps the current and voltage sensed by the power sensor 23 from excessively increasing and, in response to the temperature sensed by the temperature sensor 25, controls the output of the high-frequency in such a manner that the electrode 13 is operated within a temperature range of 10-90° C.±4.

[Multi-Channel Activation Method]

This method is utilized in fulgurating, e.g., cancer cells of a relatively large size, with a plural number of electrodes.

Initially, the user manipulates the key part 3 to set the voltage, current and impedance of the high-frequency which is to be supplied to the electrodes 13, 14 and 15 and then selects the first to third amplifying channels 10, 11 and 12 with the use of the channel selection part 4.

Under this state, if a specific key of the key part 3 is pushed by the user, the oscillator 16 generates a high-frequency of, e.g., 480 KHz, which in turn is supplied to the waveform modulator part 17. The waveform modulator part 17 modulates the waveform of the high-frequency into a form suitable for use in the electrodes 13, 14 and 15 and then supplies the high-frequency to the respective phase control parts 20 of the first to third amplifying channels 10, 11 and 12. The phase control parts 20 serve to adjust the phase of the high-frequency prior to the latter being supplied to subsequent elements.

The preamplifiers 21a and the main amplifiers 21b, which are serially connected to the phase control parts 20, function to amplify the signals of the high-frequency to have a root mean square (RMS) output of, e.g., 30-200 Watts and a load of, e.g., about 50Ω. The signals of the high-frequency are then fed to the electrodes 13, 14 and 15 through the corresponding relays 22.

At this time, the microcontroller unit 5 controls the phase control operation of the phase control parts 20 and the amplification ratio of the preamplifiers 21a and the main amplifiers 21b. The microcontroller unit 5 also controls the first to third amplifying channels 10, 11 and 12 in such a manner that they can generate the output for a time period set and measured by the timer 2. Each of the impedance matching parts 24 disposed between the main amplifiers 21b and the relays 22 serve to match the impedance on an output side of the main amplifiers 21b with the impedance on a load side of the relays 22.

In this manner, the high-frequency is applied to the electrodes 13, 14 and 15 selected by the user. If the electrodes 13 are kept in contact with or inserted into the lump of cancer cells, the cancer cells are fulgurated by the heat generated in the electrodes 13, 14 and 15.

Assuming that the electrode 14 is inserted into the center of a large-sized cancer cell lump with the electrodes 13 and 15 remaining in contact with the periphery thereof, the voltage level of the high-frequency supplied to the electrode 14 is controlled to become higher than the voltage levels to the remaining electrodes 13 and 15, as illustrated in FIG. 3. This is because the electrode 14 is responsible for treatment of a broader area of the cancer cell lump.

The microcontroller unit 5 may control the pulse timing of the high-frequency supplied to the respective electrodes 13, 14 and 14 either in an alternating pattern as shown in FIG. 3 or in a synchronized pattern (not shown).

Meanwhile, during the time when the high-frequency is supplied to the electrodes 13, 14 and 15, the power sensor 23 detects the current and voltage of the high-frequency and then feeds back the sensed current and voltage to the microcontroller unit 5. Furthermore, the temperature sensor 25 detects the temperature of the sensor 13 and then feeds back the sensed temperature to the microcontroller unit 5.

In addition, the microcontroller unit 5 keeps the current and voltage sensed by the power sensor 23 from excessively increasing and, in response to the temperature sensed by the temperature sensor 25, controls the output of the high-frequency in such a manner that the electrodes 13, 14 and 15 are operated within a temperature range of 10-90° C.±4.

As set forth above, the multi-channel activation method can be effectively used at the time when a large-sized cancer cell lump is to be fulgurated. Such a treatment process is easy to perform because a plural number of electrodes are activated by a single radio frequency generator.

As an alternative example, the radio frequency generator 1 may be used in combination with an additional radio frequency generator 1a, particularly when the size of a cancer cell lump is great enough to require the combined use of more than three electrodes at one time.

More specifically, as shown in FIG. 5, an additional radio frequency generator 1a is connected to the control communication part 8 of the radio frequency generator 1, at which time the radio frequency generator 1 serves as a master generator while the radio frequency generator 1a functions as a slave generator, and vice versa.

In other words, the microcontroller unit 5 of the master generator 1 controls the microcontroller unit (not shown) of the slave generator 1a so that a high-frequency of a desired voltage level can be supplied to the electrodes of the slave generator 1a.

Referring to FIG. 5, the electrodes 13, 14 and 15 connected to the first to third amplifying channels 10, 11 and 12 of the master generator 1 are activated by the high-frequency which is supplied under the control of the microcontroller unit 5 of the master generator 1. Likewise, the electrodes 13a, 14a and 15a connected to fourth to sixth amplifying channels 10a, 11a and 12a of the slave generator 1a are activated by the high-frequency which is supplied under the control of the microcontroller unit 5 of the master generator 1.

Such a combined use of two radio frequency generators makes it possible to treat a large-sized cancer cell lump or remotely located cancer cell lumps at one time with six electrodes. It would also be possible to activate more than six electrodes by interconnecting three or more radio frequency generators with a communication cable.

Referring back to FIG. 1, the data communication part 7 serves to notify a remote computer of the output characteristics of the high-frequency outputted from the respective amplifying channels 10, 11 and 12 so that the user can monitor the operating condition of the radio frequency generator.

As described in the foregoing, the multi-channel radio frequency generator according to the present invention can supply a high-frequency to a plurality of electrodes to thereby efficiently fulgurate large-sized cancer cells occupying an increased area with enhanced safety. Furthermore, the multi-channel radio frequency generator has an ability to control more than one amplifying channels to perform simultaneous treatments for remotely located lesions.

Although one preferred embodiment of the present invention has been described in detail, it will be apparent to those skilled in the art that various changes or modifications may be made thereto within the scope of the invention defined by the appended claims.

What is claimed is:

1. A multi-channel radio frequency generator for high-frequency thermal treatment used in combination with a plurality of electrodes, comprising:
an oscillator for producing a high-frequency;
a waveform modulator part for modulating a waveform of the high-frequency generated by the oscillator;
first to third amplifying channels for amplifying the high-frequency outputted from the waveform modulator part to have a root mean square output of 30-200 Watts and then supplying the root mean square output to the electrodes;
a channel selection part for allowing a user to select one or more of the first to third amplifying channels;
a key part for enabling the user to set a voltage, a current and an impedance for the first to third amplifying channels; and
a microcontroller unit for controlling a power, a time and a phase of the high-frequency outputted from each of the first to third amplifying channels.

2. The multi-channel radio frequency generator as recited in claim 1, wherein each of the first to third amplifying channels comprises:
a phase control part for controlling a phase of the high-frequency outputted from the waveform modulator part;
a preamplifier and a main amplifier for amplifying in two stages the high-frequency outputted from the waveform modulator part to have a root mean square output of 30-200 Watts and a load of about 50Ω; and
a relay for supplying the high-frequency outputted from the main amplifier to the electrodes.

3. The multi-channel radio frequency generator as recited in claim 2, wherein each of the first to third amplifying channels further comprises:
a power sensor for sensing a current and a voltage of the high-frequency supplied to the relay and supplying the sensed current and voltage to the microcontroller unit;
an impedance matching part for matching an impedance on an output side of the main amplifier with an impedance on a load side of the relay; and
a temperature sensor for sensing a temperature of the electrodes and supplying the sensed temperature to the microcontroller unit,
the microcontroller unit adapted to control amplification ratios of the preamplifier and the main amplifier in such a manner that the current and the voltage sensed by the power sensor are kept from increasing above predetermined values and further adapted to, in response to the temperature sensed by the temperature sensor, control the high-frequency outputted from each of the first to third amplifying channels in such a manner that the electrodes are operated within a temperature range of 10-90° C.±4.

4. The multi-channel radio frequency generator as recited in claim 1, further comprising:
a timer for setting and measuring an operation time of each of the first to third amplifying channels;
a display part for displaying information detected by sensors of the first to third amplifying channels on a channel-by-channel basis;
a data communication part for notifying a remote computer of an operating condition of the radio frequency generator so that the user can monitor the operating condition of the radio frequency generator; and
a control communication part to which one or more additional radio frequency generator is connected for use in combination.

5. The multi-channel radio frequency generator as recited in claim 4, wherein the additional radio frequency generator is used as a slave radio frequency generator.
